(19) United States
(12) Patent Application Publication
(10) Pub. No.: US 2010/0040691 A1
(43) Pub. Date: Feb. 18, 2010

(54) PHARMACEUTICAL COMPOSITIONS COMPRISING METHOTREXATE

(75) Inventors: Andrew John McGlashan Richards, Cambridgeshire (GB); Peter Strong, Hertfordshire (GB); Ann Gail Hayes, Hertfordshire (GB); David Alexander Vodden Morton, Poole Dorset (GB)

Correspondence Address: Davidson, Davidson & Kappel, LLC, 485 7th Avenue, 14th Floor, New York, NY, 10018, US

(73) Assignee: Vectura Group PLC, Wiltshire (GB)

(21) Appl. No.: 12/085,108

(22) PCT Filed: Nov. 17, 2006
(86) PCT No.: PCT/GB2006/050397

§ 371 Date: Oct. 30, 2009

(30) Foreign Application Priority Data
Nov. 18, 2005 (GB) 0523576.7

(51) Int. Cl. A61K 31/519; A61K 9/12; A61K 31/56; C07D 475/08; A61P 11/06; A61P 11/12; A61P 29/00; A61K 9/14

(52) U.S. Cl. 424/489; 424/45; 514/171; 514/249; 544/260

(57) ABSTRACT

The present invention relates to pharmaceutical compositions and their uses in therapy. In particular, the invention relates to compositions comprising methotrexate, preferably wherein the compositions are for administration via the inhaled or intranasal route.

The present invention relates to pharmaceutical compositions and their uses in therapy. In particular, the invention relates to compositions comprising methotrexate, preferably wherein the compositions are for administration via the inhaled or intranasal route.

Methotrexate is an antimetabolite drug which has been used in the treatment of certain diseases associated with abnormally rapid cell growth, including cancer and autoimmune diseases such as breast cancer and psoriasis. Currently, methotrexate is probably most widely used for treating rheumatoid arthritis, although its mechanism of action in this illness is not known. The principle mode of action for Methotrexate is to provide anti-inflammatory action for both the pulmonary and nasal airways.

Methotrexate is currently provided in the form of compositions for oral administration or for subcutaneous, intramuscular, intravenous or intrathecal injection. This administration of methotrexate provides a systemic effect.

Patients generally receive weekly doses rather than daily, in an attempt to decrease the risk of certain side effects. Side effects include anaemia, neutropenia, increased risk of bruising, nausea and vomiting, dermatitis and diarrhoea.

In addition, methotrexate has been associated with a number of serious pulmonary side effects. Pulmonary toxicity of methotrexate has been well-described and may take a variety of forms. Pulmonary infiltrates are a commonly encountered problem and these infiltrates resemble hypersensitivity lung disease (Expert Opin Drug Saf. 2005 July; 4(4):723-30). Methotrexate-induced pneumonitis has also been recognised as being a serious and unpredictable clinical problem. Whilst the mechanism of this side effect remains largely unclear, it is possible that the methotrexate triggers the release of IL-8, G-CSF, MCP-1, GM-CSF, and LTB(4), which may play an important role methotrexate-induced lung inflammation (Clin Sci (Lond) 2004 June; 106(6):619-25 and Exp Lung Res. 2003 March; 29(2):91-111). There have also been reports of methotrexate-induced noncardiogenic pulmonary edema in patients receiving high doses of methotrexate for anti-cancer therapy (Intern Med. 2004 September; 43(9):846-51). It has been reported that, if given in high doses, methotrexate can cause pulmonary complications, with a significant reduction in percent predicted values of forced expiratory volume (FEV₁), forced vital capacity (FVC), total lung capacity (TLC), and functional residual capacity (FRC) having been observed after 2 years of methotrexate treatment for rheumatoid arthritis (Rheumatol Int. 2002 September; 22(5):204-7. Epub 2002 Jul 16).

Chronic respiratory disease can be associated with evidence of systemic inflammatory changes. In 2004 Gan et al, published a review suggesting that increased levels of systemic inflammatory markers were associated with reduced lung function in COPD patients (Thorax 2004, 59, 574-580). More recently it has been suggested that there is an inverse relationship between pulmonary function and C-reactive protein levels in apparently healthy people (Am J Resp Care Crit Med 2006, 174, 626-632).

At present, however, the limited amount of work in the literature does not clarify whether the elevated levels of systemic inflammatory markers including CRP, are a secondary consequence of on-going inflammation in the lungs or a systemic effect. Patients that present with elevated levels of plasma C-reactive protein, or other systemic inflammatory markers may, therefore, benefit from treatment with low doses of inhaled Methotrexate which act exclusively, or if not predominantly, in the lung.

Chronic respiratory diseases, including sarcoidosis, chronic obstructive pulmonary disease (COPD), cystic fibrosis (CF) and asthma constitute a major health problem, but are poorly treated by current therapies. These conditions involve inflammation of the airways and known therapies include inhaled corticosteroids. However, these are not always efficacious and the chronic use of such steroids may give rise to unacceptable side effects, including systemic side effects.

Sleep apnoea is a condition in which sufferers stop breathing when asleep and it is now recognised to cause a range of serious health complications, including sleepiness during daytime hours. In obstructive sleep apnoea, the apnoea is triggered by the upper airway becoming blocked during sleep. Recent published research has produced evidence that obstructive sleep apnoea can be associated with inflammation in both the upper and lower airways. The most commonly used agents to treat inflammation in the upper airways are intranasal steroids. However, to date the literature provides no clear conclusions regarding the role of intranasal steroids in the treatment of obstructive airways disease or the precise role of airway inflammation in the disease.

According to a first aspect of the present invention, a composition comprising methotrexate is provided, wherein the composition is for pulmonary or intranasal administration to provide a therapeutic effect.

The methotrexate used in these compositions can be in any suitable form, including salts, isomers; prodrugs and active metabolites of methotrexate.

In one embodiment, the compositions according to the invention are for treating inflammation, and especially for treating inflammation of the airways. This inflammation may be of the upper or lower airways, or both.

In particular, the compositions according to the present invention may be used to treat inflammation associated with chronic respiratory diseases such as sarcoidosis, chronic obstructive pulmonary disease (COPD), cystic fibrosis (CF), asthma, obstructive sleep apnoea or any combination thereof.

The disclosure in the past of numerous and serious pulmonary side effects associated with oral or injected methotrexate would certainly discourage the skilled person from considering pulmonary or intranasal administration of methotrexate.

However, whilst methotrexate has previously been used to provide a systemic effect, administering the drug via the pulmonary or intranasal route means that it is possible to now use methotrexate to provide a local effect. Benefits associated with this are faster, more effective treatment, smaller doses and consequently fewer side effects.

When used to treat respiratory disorders, such as the chronic respiratory diseases mentioned above, the compositions according to the present invention are preferably administered by inhalation, but may also involve intranasal delivery.

According to one embodiment, the composition is a dry powder for pulmonary, administration by inhalation. Preferably, such dry powder compositions are dispensed using a dry powder inhaler (DPI).

The compositions according to the present invention may be administered using active or passive DPIs. As it has now been identified how one may tailor a dry powder formulation to the specific type of device used to dispense it, this means that the perceived disadvantages of passive devices where high performance is sought may be overcome.

Preferably, for delivery to the lower respiratory tract or deep lung, the mass median aerodynamic diameter (MMAD) of the active particles in a dry powder composition is not more than 10 μm, and preferably not more than 5 μm, more preferably not more than 3 μm, and may be less than 2 μm, less than 1.5 μm or less than 1 μm. Especially for deep lung or systemic delivery, the active particles may have a size of 0.1 to 3 μm or 0.1 to 2 μm.

Ideally, at least 90% by weight of the active particles in a dry powder formulation should have an aerodynamic diameter of not more than 10 μm, preferably not more than 5 μm, more preferably not more than 3 μm, not more than 2.5 μm, not more than 2.0 μm, not more than 1.5 μm, or even not more than 1.0 μm.

When dry powders are produced using conventional processes, the active particles will vary in size, and often this variation can be considerable. This can make it difficult to ensure that a high enough proportion of the active particles are of the appropriate size for administration to the correct site. In certain circumstances it may therefore be desirable to have a dry powder formulation wherein the size distribution of the active particles is narrow. For example, the geometric standard deviation of the active particle aerodynamic or volumetric size distribution (σg), may preferably be not more than 2, more preferably not more than 1.8, not more than 1.6, not more than 1.5, not more than 1.4, or even not more than 1.2. A narrow particle size distribution may be of particular importance in view of methotrexate's narrow therapeutic index. A narrow particle size ensures that doses are both reproducible with respect to methotrexate content and that the dose is delivered to the same region of the lung on each delivery ensuring a reproducible pharmacokinetic profile. This may improve dose efficiency and reproducibility.

Fine particles, that is, those with an MMAD of less than 10 μm and smaller, tend to be increasingly thermodynamically unstable as their surface area to volume ratio increases, which provides an increasing surface free energy with this decreasing particle size, and consequently increases the tendency of particles to agglomerate and the strength of the agglomerate. In the inhaler, agglomeration of fine particles and adherence of such particles to the walls of the inhaler are problems that result in the fine particles leaving the inhaler as large, stable agglomerates, or being unable to leave the inhaler and remaining adhered to the interior of the inhaler, or even clogging or blocking the inhaler.

The uncertainty as to the extent of formation of stable agglomerates of the particles between each actuation of the inhaler, and also between different inhalers and different batches of particles, leads to poor dose reproducibility. Furthermore, the formation of agglomerates means that the MMAD of the active particles can be vastly increased, with agglomerates of the active particles not reaching the required part of the lung.

In an attempt to improve this situation and to provide a consistent FPF and FPD, dry powder formulations often include additive material. The additive material is intended to control the cohesion between particles in the dry powder formulation. It is thought that the additive material interferes with the weak bonding forces between the small particles, helping to keep the particles separated and reducing the adhesion of such particles to one another, to other particles in the formulation if present and to the internal surfaces of the inhaler device. Where agglomerates of particles are formed, the addition of particles of additive material decreases the stability of those agglomerates so that they are more likely to break up in the turbulent air stream created on actuation of the inhaler device, whereupon the particles are expelled from the device and inhaled. As the agglomerates break up, the active particles return to the form of small individual particles which are capable of reaching the lower lung.

However, the optimum stability of agglomerates to provide efficient drug delivery will depend upon the nature of the turbulence created by the particular device used to deliver the powder. Agglomerates will need to be stable enough for the powder to exhibit good flow characteristics during processing and loading into the device, whilst being unstable enough to release the active particles of respirable size upon actuation.

Preferably, the additive material is an anti-adherent material and it will tend to reduce the cohesion between particles and will also prevent fine particles becoming attached to the inner surfaces of the inhaler device. Advantageously, the additive material is an anti-friction agent or glidant and will give better flow of the pharmaceutical composition in the inhaler. The additive materials used in this way may not necessarily be usually referred to as anti-adherents or anti-friction agents, but they will have the effect of decreasing the cohesion between the particles or improving the flow of the powder. The additive materials are often referred to as force control agents (FCAs) and they usually lead to better dose reproducibility and higher fine particle fractions. Therefore, a FCA, as used herein, is an agent whose presence on the surface of a particle can modify the adhesive and cohesive surface forces experienced by that particle, in the presence of other particles. In general, its function is to reduce both the adhesive and cohesive forces.

Known FCAs usually consist of physiologically acceptable material although the additive material may not always reach the lung. Preferred materials for used in dry powder compositions include amino acids, peptides and polypeptides having a molecular weight of between 0.25 and 1000 kDa and derivatives thereof.

It is particularly advantageous for the FCA to comprise an amino acid. The FCA may comprise or consist of one or more of any of the following amino acids: leucine, isoleucine, lysine, valine, methionine, and phenylalanine. The FCA may be a salt or a derivative of an amino acid, for example aspartame or acesulfame K. Preferably, the FCA consists substantially of an amino acid, more preferably of leucine, advantageously L-leucine. The D- and DL-forms may also be used. The FCA may comprise Aerocine™, amino acid particles as disclosed in the earlier patent application published as WO 00/33811.

The FCA may comprise or consist of dipolar ions, which may be zwitterions. It is also advantageous for the FCA to comprise or consist of a spreading agent, to assist with the dispersal of the composition in the lungs. Suitable spreading agents include surfactants such as known lung surfactants (e.g. ALEC®) which comprise phospholipids, for example, mixtures of DPPC (dipalmitoyl phosphatidylcholine) and PG (phosphatidylglycerol). Other suitable surfactants include, for example, dipalmitoyl phosphatidylethanolamine (DPPE), dipalmitoyl phosphatidylinositol (DPPI).

The FCA may comprise or consist of a metal stearate, for example, zinc stearate, magnesium stearate, calcium stearate, sodium stearate or lithium stearate, or a derivative thereof, for example, sodium stearyl fumarate or sodium stearyl lactylate.

The FCA may comprise or consist of one or more surface active materials, in particular materials that are surface active in the solid state, which may be water soluble or water dispersible, for example lecithin, in particular soya lecithin, or substantially water insoluble, for example solid state fatty acids such as oleic acid, lauric acid, palmitic acid, stearic acid, erucic acid, behenic acid, or derivatives (such as esters and salts) thereof, such as glyceryl behenate. Specific examples of such surface active materials are phosphatidylcholines, phosphatidylethanolamines, phosphatidylglycerols and other examples of natural and synthetic lung surfactants; lauric acid and its salts, for example, sodium lauryl sulphate, magnesium lauryl sulphate; triglycerides such as Dynsan 118 and Cutina HR; and sugar esters in general. Alternatively, the FCA may comprise or consist of cholesterol. Other useful FCAs are film-forming agents, fatty acids and their derivatives, as well as lipids and lipid-like materials.

Other possible FCAs include sodium benzoate, hydrogenated oils which are solid at room temperature, talc, titanium dioxide, aluminium dioxide, silicon dioxide and starch.

In some embodiments, a plurality of different FCAs can be used.

Dry powder compositions often include carrier particles mixed with fine particles of active material. In such compositions, rather than sticking to one another, the fine active particles tend to adhere to the surfaces of the carrier particles whilst in the inhaler device, but are supposed to release and become dispersed upon actuation of the dispensing device and inhalation into the respiratory tract, to give a fine suspension. Such release may be improved by the inclusion of an FCA.

Carrier particles may comprise or consist of any acceptable excipient material or combination of materials and preferably the material(s) is (are) inert and physiologically acceptable. For example, the carrier particles may be composed of one or more materials selected from sugar alcohols, polyols and crystalline sugars. Other suitable carriers include inorganic salts such as sodium chloride and calcium carbonate, organic salts such as sodium lactate and other organic compounds such as polysaccharides and oligosaccharides. Advantageously the carrier particles are of a polyol. In particular the carrier particles may be particles of crystalline sugar, for example mannitol, dextrose or lactose. Preferably, the carrier particles are of lactose.

According to some embodiments of the present invention, the dry powder compositions include carrier particles that are relatively large, compared to the particles of active material. This means that substantially all (by weight) of the carrier particles have a diameter which lies between 20 μm and 1000 μm, or between 50 μm and 1000 μm. Preferably, the diameter of substantially all (by weight) of the carrier particles is less than 355 μm and lies between 20 μm and 250 μm. In one embodiment, the carrier particles have a MMAD of at least 90 μm.

Preferably, at least 90% by weight of the carrier particles have a diameter between from 60 μm to 180 μm. The relatively large diameter of the carrier particles improves the opportunity for other, smaller particles to become attached to the surfaces of the carrier particles and to provide good flow and entrainment characteristics and improved release of the active particles in the airways to increase deposition of the active particles in the lower lung.

Powder flow problems associated with compositions comprising larger amounts of fine material, such as up to from 5 to 20% by total weight of the formulation. This problem may be overcome by the use of large fissured lactose carrier particles, as is discussed in earlier patent applications published as WO 01/78694, WO 01/78695 and WO 01/78696.

In other embodiments, the excipient or carrier particles included in the dry powder compositions are relatively small, having a median diameter of about 3 to about 40 μm, preferably about 5 to about 30 μm, more preferably about 5 to about 20 μm, and most preferably about 5 to about 15 μm. Such fine carrier particles, if untreated with an additive are unable to provide suitable flow properties when incorporated in a powder composition comprising fine or ultra-fine active particles. Indeed, previously, particles in these size ranges would not have been regarded as suitable for use as carrier particles, and instead would only have been added in small quantities as a fine component in combination with coarse carrier particles, in order to increase the aerosolisation properties of compositions containing a drug and a larger carrier, typically with median diameter 40 μm to 100 μm or greater. However, the quantity of such a fine excipient may be increased and such fine excipient particles may act as carrier particles if these particles are treated with an additive or FCA, even in the absence of coarse carrier particles. Such treatment can bring about substantial changes in the powder characteristics of the fine excipient particles and the powders they are included in. Powder density is increased, even doubled, for example from 0.3 g/cc to over 0.5 g/cc. Other powder characteristics are changed, for example, the angle of repose is reduced and contact angle increased.

Treated fine carrier particles having a median diameter of 3 to 40 μm are advantageous as their relatively small size means that they have a reduced tendency to segregate from the drug component, even when they have been treated with an additive to reduce cohesion. This is because the size differential between the carrier and drug is relatively small compared to that in conventional compositions which include fine or ultra-fine active particles and much larger carrier particles. The surface area to volume ratio presented by the fine carrier particles is correspondingly greater than that of conventional large carrier particles. This higher surface area, allows the carrier to be successfully associated with higher levels of drug than for conventional larger carrier particles. This makes the use of treated fine carrier particles particularly attractive in powder compositions to be dispensed by passive devices.

The metered dose (MD) of a dry powder composition is the total mass of active agent present in the metered form presented by the inhaler device in question. For example, the MD might be the mass of active agent present in a capsule for a Cyclohaler™, or in a foil blister in a Gyrohaler™ device.

The emitted dose (ED) is the total mass of the active agent emitted from the device following actuation. It does not include the material left on the internal or external surfaces of the device, or in the metering system including, for example, the capsule or blister. The ED is measured by collecting the total emitted mass from the device in an apparatus frequently identified as a dose uniformity sampling apparatus (DUSA), and recovering this by a validated quantitative wet chemical assay (a gravimetric method is possible, but this is less precise).

The fine particle dose (FPD) is the total mass of active agent which is emitted from the device following actuation which is present in an aerodynamic particle size smaller than a defined limit. This limit is generally taken to be 5 μm if not expressly stated to be an alternative limit, such as 3 μm, 2 μm or 1 μm, etc. The FPD is measured using an impactor or impinger, such as a twin stage impinger (TSI), multi-stage impinger (MSI), Andersen Cascade Impactor (ACI) or a Next Generation Impactor (NGI). Each impactor or impinger has a pre-determined aerodynamic particle size collection cut points for each stage. The FPD value is obtained by interpretation of the stage-by-stage active agent recovery quantified by a validated quantitative wet chemical assay (a gravimetric method is possible, but this is less precise) where either a simple stage cut is used to determine FPD or a more complex mathematical interpolation of the stage-by-stage deposition is used.

The fine particle fraction (FPF) is normally defined as the FPD divided by the ED and expressed as a percentage. Herein, the FPF of ED is referred to as FPF(ED) and is calculated as FPF(ED)=(FPD/ED)×100%.

The fine particle fraction (FPF) may also be defined as the FPD divided by the MD and expressed as a percentage. Herein, the FPF of MD is referred to as FPF(MD), and is calculated as FPF(MD)=(FPD/MD)×100%.

In one embodiment of the invention, the composition is a dry powder which has a fine particle fraction (<5 μm) of at least 50%, preferably at least 60%, at least 70% or at least 80%.

Preferably, these FPFs are achieved when the composition is dispensed using an active DPI, although such good FPFs may also be achieved using passive DPIs, especially where the device is one as described in the earlier patent application published as WO 2005/037353 and/or the dry powder composition has been formulated specifically for administration by a passive device.

In one embodiment of the invention, the DPI is an active device, in which a source of compressed gas or alternative energy source is used. Examples of suitable active devices include Aspirair™ (Vectura Ltd) and the active inhaler device produced by Nektar Therapeutics (as disclosed in U.S. Pat. No. 6,257,233), and the ultrasonic Microdose™ or Oriel™ devices.

In an alternative embodiment, the DPI is a passive device, in which the patient's breath is the only source of gas which provides a motive force in the device. Examples of “passive” dry powder inhaler devices include the Rotahaler™ and Diskhaler™ (GlaxoSmithKline) and the Turbohaler™ (Astra-Draco) and Novolizer™ (Viatris GmbH) and GyroHaler™ (Vectura).

The dry powder formulations may be pre-metered and kept in capsules or foil blisters which offer chemical and physical protection whilst not being detrimental to the overall performance. Alternatively, the dry powder formulations may be held in a reservoir-based device and metered on actuation. Examples of “reservoir-based” inhaler devices include the Clickhaler™ (Innovata) and Duohaler™ (Innovata), and the Turbohaler™ (Astra-Draco). Actuation of such reservoir-based inhaler devices can comprise passive actuation, wherein the patient's breath is the only source of energy which generates a motive force in the device.

The particles of active agent included in the compositions of the present invention, may be formulated with additional excipients to aid delivery or to control release of the active agent upon deposition within the lung. In such embodiments, the active agent may be embedded in or dispersed throughout particles of an excipient material which may be, for example, a polysaccharide matrix. Alternatively, the excipient may form a coating, partially or completely surrounding the particles of active material. Upon delivery of these particles to the lung, the excipient material acts as a temporary barrier to the release of the active agent, providing a delayed or sustained release of the active agent. Suitable excipient materials for use in delaying or controlling the release of the active material will be well known to the skilled person and will include, for example, pharmaceutically acceptable soluble or insoluble materials such as polysaccharides, for example xanthan gum. A dry powder composition may comprise the active agent in the form of particles which provide immediate release, as well as particles exhibiting delayed or sustained release, to provide any desired release profile.

Compositions according to the invention may be produced using conventional formulation techniques.

Spray drying is a well-known and widely used technique for producing particles of active material of inhalable size. Conventional spray drying techniques may be improved so as to produce active particles with enhanced chemical and physical properties so that they perform better when dispensed from a DPI than particles formed using conventional spray drying techniques. Such improvements are described in detail in the earlier patent application published as WO 2005/025535.

In particular, it is disclosed that co-spray drying an active agent with an FCA under specific conditions can result in particles with excellent properties which perform extremely well when administered by a DPI for inhalation into the lung.

It has been found that manipulating or adjusting the spray drying process can result in the FCA being largely present on the surface of the particles. That is, the FCA is concentrated at the surface of the particles, rather than being homogeneously distributed throughout the particles. This clearly means that the FCA will be able to reduce the tendency of the particles to agglomerate. This will assist the formation of unstable agglomerates that are easily and consistently broken up upon actuation of a DPI.

It has been found that it may be advantageous to control the formation of the droplets in the spray drying process, so that droplets of a given size and of a narrow size distribution are formed. Furthermore, controlling the formation of the droplets can allow control of the air flow around the droplets which, in turn, can be used to control the drying of the droplets and, in particular, the rate of drying. Controlling the formation of the droplets may be achieved by using alternatives to the conventional 2-fluid nozzles, especially avoiding the use of high velocity air flows. In particular, it is preferred to use a spray drier comprising a means for producing droplets moving at a controlled velocity and of a predetermined droplet size. The velocity of the droplets is preferably controlled relative to the body of gas into which they are sprayed. This can be achieved by controlling the droplets' initial velocity and/or the velocity of the body of gas into which they are sprayed, for example by using an ultrasonic nebuliser (USN) to produce the droplets. Alternative nozzles such as electrospray nozzles or vibrating orifice nozzles may be used.

Spray drying may be used to produce the microparticles comprising the methotrexate. The spray drying process may be adapted to produce spray-dried particles that include the active agent dispersed or suspended within a material that provides the controlled release properties.

The process of milling, for example, jet milling, may also be used to formulate the dry powder compositions according to the present invention. The manufacture of fine particles by milling can be achieved using conventional techniques. In the conventional use of the word, “milling” means the use of any mechanical process which applies sufficient force to the particles of active material that it is capable of breaking coarse particles (for example, particles with a MMAD greater than 100 μm) down to fine particles (for example, having a MMAD not more than 50 μm). In the present invention, the term “milling” also refers to deagglomeration of particles in a formulation, with or without particle size reduction. The particles being milled may be large or fine prior to the milling step. A wide range of milling devices and conditions are suitable for use in the production of the compositions of the inventions. The selection of appropriate milling conditions, for example, intensity of milling and duration, to provide the required degree of force will be within the ability of the skilled person. Ball milling is a preferred method. Alternatively, a high pressure homogeniser may be used in which a fluid containing the particles is forced through a valve at high pressure producing conditions of high sheer and turbulence. Sheer forces on the particles, impacts between the particles and machine surfaces or other particles, and cavitation due to acceleration of the fluid may all contribute to the fracture of the particles. Suitable homogenisers include the EmulsiFlex high pressure homogeniser, the Niro Soavi high pressure homogeniser and the Microfluidics Microfluidiser. The milling process can be used to provide the microparticles with mass median aerodynamic diameters as specified above.

Milling the active agent with a force control agent and/or with a material which can delay or control the release of the active agent is preferred. Co-milling or co-micronising particles of active agent and particles of FCA or excipient will result in the FCA or excipient becoming deformed and being smeared over or fused to the surfaces of fine active particles. These resultant composite active particles comprising an FCA have been found to be less cohesive after the milling treatment. If a significant reduction in particle size is also required, co-jet milling is preferred, as disclosed in the earlier patent application published as WO 2005/025536. The co-jet milling process can result in composite active particles with low micron or sub-micron diameter, and these particles exhibit particularly good FPF and FPD, even when dispensed using a passive DPI.

The milling processes apply a high enough degree of force to break up tightly bound agglomerates of fine or ultra-fine particles, such that effective mixing and effective application of the additive material to the surfaces of those particles is achieved.

The co-milling or co-micronising of active and additive particles may involve compressive type processes, such as mechanofusion, cyclomixing and related methods such as those involving the use of a Hybridiser or the Nobilta. The principles behind these processes are distinct from those of alternative milling techniques in that they involve a particular interaction between an inner element and a vessel wall, and in that they are based on providing energy by a controlled and substantial compressive force, preferably compression within a gap of predetermined width.

In one embodiment, if required, the microparticles produced by the milling step can then be formulated with an additional excipient. This may be achieved by a spray drying process, e.g. co-spray drying. In this embodiment, the particles are suspended in a solvent and co-spray dried with a solution or suspension of the additional excipient. Preferred additional excipients include polysaccharides. Additional pharmaceutical effective excipients may also be used.

In a yet further embodiment, the composition is a solution or suspension and is administered using a pressurised metered dose inhaler (pMDI), a nebuliser or a soft mist inhaler. Examples of suitable devices include pMDIs such as Modulite® (Chiesi), SkyeFine™ and SkyeDry™ (SkyePharma). Nebulisers such as Porta-Neb®, Inquaneb™ (Pari) and Aquilon™, and soft mist inhalers such as eFlow™ (Pari), Aerodose™ (Aerogen), Respimat® Inhaler (Boehringer Ingelheim GmbH), AERx® Inhaler (Aradigm) and Mystic™ (Ventaira Pharmaceuticals, Inc.).

Where the composition is to be dispensed using a pMDI, the composition comprising methotrexate preferably further comprises a propellant. In embodiments of the present invention, the propellant is CFC-12 or an ozone-friendly, non-CFC propellant, such as 1,1,1,2-tetrafluoroethane (HFC 134a), 1,1,1,2,3,3,3-heptafluoropropane (HFC-227), HCFC-22 (difluororchloromethane), HFA-152 (difluoroethane and isobutene) or combinations thereof. Such formulations may require the inclusion of a polar surfactant such as polyethylene glycol, diethylene glycol monoethyl ether, polyoxyethylene sorbitan monolaurate, polyoxyethylene sorbitan monooleate, propoxylated polyethylene glycol, and polyoxyethylene lauryl ether for suspending, solubilizing, wetting and emulsifying the active agent and/or other components, and for lubricating the valve components of the MDI.

Where the composition is to be dispensed using a nebuliser or soft mist inhaler, the composition is in the form of a solution or suspension. Thus, in some embodiments, these compositions comprise a solvent and/or water.

In one embodiment, an ultrasonic nebuliser (USN) is used to form the droplets in the spray mist. USNs use an ultrasonic transducer which is submerged in a liquid. The ultrasonic transducer (a piezoelectric crystal) vibrates at ultrasonic frequencies to produce the short wavelengths required for liquid atomisation. In one common form of USN, the base of the crystal is held such that the vibrations are transmitted from its surface to the nebuliser liquid, either directly or via a coupling liquid, which is usually water. When the ultrasonic vibrations are sufficiently intense, a fountain of liquid is formed at the surface of the liquid in the nebuliser chamber. Droplets are emitted from the apex and a “fog” emitted.

Whilst ultrasonic nebulisers (USNs) are known, these are conventionally used in inhaler devices, for the direct inhalation of solutions containing drug, and they have not previously been widely used in a spray drying apparatus. It has been discovered that the use of such a nebuliser in spray drying has a number of important advantages and these have not previously been recognised. The preferred USNs control the velocity of the particles and therefore the rate at which the particles are dried, which in turn affects the shape and density of the resultant particles. The use of USNs also provides an opportunity to perform spray drying on a larger scale than is possible using conventional spray drying apparatus with conventional types of nozzles used to create the droplets, such as 2-fluid nozzles.

The attractive characteristics of USNs for producing fine particle dry powders include: low spray velocity; the small amount of carrier gas required to operate the nebulisers; the comparatively small droplet size and narrow droplet size distribution produced; the simple nature of the USNs (the absence of moving parts which can wear, contamination, etc.); the ability to accurately control the gas flow around the droplets, thereby controlling the rate of drying; and the high output rate which makes the production of dry powders using USNs commercially viable in a way that is difficult and expensive when using a conventional two-fluid nozzle arrangement.

USNs do not separate the liquid into droplets by increasing the velocity of the liquid. Rather, the necessary energy is provided by the vibration caused by the ultrasonic nebuliser.

In one embodiment of the present invention, the composition further includes one or more other pharmaceutically active agent, and preferably an agent which is useful in the treatment of respiratory disorders. Such agents include bronchodilators, such as β₂-agonists, such as bambuterol, bitolterol, fenoterol, formoterol, levalbuterol, metaproterenol, pirbuterol, procaterol, salbutamol, salmeterol, terbutaline and the like; antimuscarinics such as ipratropium, ipratropium bromide, tiotropium, LAS-34273, glycopyrronium, glycopyrrolate and the like; xanthines such as aminophylline, theophylline and the like; and other respiratory agents such as ephedrine, epinephrine, isoetharine, isoproterenol, montelukast, pseudoephedrine, sibenadet and zafirlukast.

The compositions according to the present invention may also include steroids, such as, for example, alcometasone, beclomethasone, beclomethasone dipropionate, betamethasone, budesonide, ciclesonide, clobetasol, deflazacort, diflucortolone, desoxymethasone, dexamethasone, fludrocortisone, flunisolide, fluocinolone, fluometholone, fluticasone, fluticasone proprionate, hydrocortisone, mometasone, methylprednisolone, nandrolone decanoate, neomycin sulphate, prednisolone, rimexolone, triamcinolone and triamcinolone acetonide.

Other types of active agents that may be included in the compositions of the present invention include: mucolytics such as N-acetylcysteine, amiloride, dextrans, heparin, desulphated heparin, low molecular weight heparin and recombinant human DNase; matrix metalloproteinase inhibitors (MMPIs); leukotriene receptor antagonists; 5-lipooxygenase inhibitors; antibiotics; antineoplastics; peptides; vaccines; antitussives; nicotine; PDE3 inhibitors; PDE4 inhibitors; mixed PDE3/4 inhibitors; elastase inhibitors; and mast cell stabilizers such as sodium cromoglycate and nedocromil.

The further active agent or agents may be included in dry powder compositions in the form of separate fine particles, or they can be in the form of composite particles also including methotrexate.

Details of the therapy according to the present invention will depend on various factors, such as the age, sex or condition of the patient, and the existence or otherwise of one or more concomitant therapies. The nature and severity of the condition will also have to be taken into account.

The compositions of the present invention enhance lung function over a prolonged period of treatment and raise FEV₁ levels. Following initial dosing, and subsequent doses, the FEV₁ level may be maintained at a higher level than that prior to the start of the therapy. The amount of methotrexate (and any other active agent included in the compositions) released over this period can be sufficient to provide effective relief of the respiratory disease, over a desired period.

Lung function may be assessed by techniques known to the skilled person, including spriometry. This may be used to measure the FEV₁ value that is greater than 10% of the predicted normal value, preferably greater than 20% and most preferably greater than 30%, over the administration period.

The size of the inhaled doses of methotrexate can vary from micrograms to tens of milligrams. In one embodiment of the invention, the composition is intended for once a week administration and the dose of methotrexate is preferably between 5 μg and 3000 μg, or between 25 μg and 500 μg.

In an alternative embodiment, the composition is intended for daily administration and the dose of methotrexate is preferably between 1 μg and 500 μg, or between 5 μg and 100 μg. When administered daily, the dose of methotrexate may be given in a single dose or divided into up to 4 doses.

Folic acid may be orally administered as a rescue therapy in the event of hepatotoxicity as a result of relatively high doses of inhaled methotrexate being delivered.

The present invention is also applicable to intranasal delivery, especially where the condition to be treated is sleep apnoea. Compositions according to the present invention are provided which are intended for this alternative mode of administration to the nasal mucosa.

Topical administration of methotrexate via intranasal administration is able to exert an anti-inflammatory effect which is complimentary to that of intranasal steroids. In patients with obstructive sleep apnoea, treatment with topical methotrexate produces a local anti-inflammatory effect which can lead to an improvement in snoring noise, sleep quality and daytime sleepiness. Treatment with topical methotrexate may be of particular help in patients whose obstructive sleep apnoea has been confirmed as being associated with inflammation of the airways.

Compositions for intranasal administration may be in the form of dry powders, solutions or suspensions.

The prior art mentions two types of processes in the context of co-milling or co-micronising active and additive particles.

First, there is the compressive type process, such as Mechano-Fusion and Cyclomix methods. As the name suggests, Mechano-Fusion is a dry coating process designed to mechanically fuse a first material onto a second material. It should be noted that the use of the terms “Mechano-Fusion” and “Mechanofused” are supposed to be interpreted as a reference to a particular type of milling process, but not a milling process performed in a particular apparatus. The first material is generally smaller and/or softer than the second. The Mechano-Fusion and Cyclomix working principles are distinct from alternative milling techniques in having a particular interaction between an inner element and a vessel wall, and are based on providing energy by a controlled and substantial compressive force.

The fine active particles and the additive particles are fed into the Mechano-Fusion driven vessel (such as a Mechano-Fusion system (Hosokawa Micron Ltd)), where they are subject to a centrifugal force and are pressed against the vessel inner wall. The powder is compressed between the fixed clearance of the drum wall and a curved inner element with high relative speed between drum and element. The inner wall and the curved element together form a gap or nip in which the particles are pressed together. As a result, the particles experience very high shear forces and very strong compressive stresses as they are trapped between the inner drum wall and the inner element (which has a greater curvature than the inner drum wall). The particles are pressed against each other with enough energy to locally heat and soften, break, distort, flatten and wrap the additive particles around the core particle to form a coating. The energy is generally sufficient to break up agglomerates and some degree of size reduction of both components may occur.

These Mechano-Fusion and Cyclomix processes apply a high enough degree of force to separate the individual particles of active material and to break up tightly bound agglomerates of the active particles such that effective mixing and effective application of the additive material to the surfaces of those particles is achieved. An especially desirable aspect of the described co-milling processes is that the additive material becomes deformed in the milling and may be smeared over or fused to the surfaces of the active particles.

However, in practice, this compression process produces little or no milling (i.e. size reduction) of the drug particles, especially where they are already in a micronised form (i.e. <10 μm), the only physical change which may be observed is a plastic deformation of the particles to a rounder shape.

Secondly, there are the impact milling processes involved in ball milling and the use of a homogenizer.

Ball milling is a suitable milling method for use in the prior art co-milling processes. Centrifugal and planetary ball milling are especially preferred methods. Alternatively, a high pressure homogeniser may be used in which a fluid containing the particles is forced through a valve at high pressure producing conditions of high shear and turbulence. Such homogenisers may be more suitable than ball mills for use in large scale preparations of the composite active particles.

Suitable homogenisers include EmulsiFlex high pressure homogenisers which are capable of pressures up to 4000 bar, Niro Soavi high pressure homogenisers (capable of pressures up to 2000 bar), and Microfluidics Microfluidisers (maximum pressure 2750 bar). The milling step may, alternatively, involve a high energy media mill or an agitator bead mill, for example, the Netzsch high energy media mill, or the DYNO-mill (Willy A. Bachofen AG, Switzerland).

These processes create high-energy impacts between media and particles or between particles. In practice, while these processes are good at making very small particles, it has been found that neither the ball mill nor the homogenizer was effective in producing dispersion improvements in resultant drug powders in the way observed for the compressive process. It is believed that the second impact processes are not as effective in producing a coating of additive material on each particle.

Conventional methods comprising co-milling active material with additive materials (as described in WO 02/43701) result in composite active particles which are fine particles of active material with an amount of the additive material on their surfaces. The additive material is preferably in the form of a coating on the surfaces of the particles of active material. The coating may be a discontinuous coating. The additive material may be in the form of particles adhering to the surfaces of the particles of active material.

At least some of the composite active particles may be in the form of agglomerates. However, when the composite active particles are included in a pharmaceutical composition, the additive material promotes the dispersal of the composite active particles on administration of that composition to a patient, via actuation of an inhaler.

Jet mills are capable of reducing solids to particle sizes in the low-micron to submicron range. The grinding energy is created by gas streams from horizontal grinding air nozzles. Particles in the fluidized bed created by the gas streams are accelerated towards the centre of the mill, colliding with slower moving particles. The gas streams and the particles carried in them create a violent turbulence and as the particles collide with one another they are pulverized.

In the past, jet-milling has not been considered attractive for co-milling active and additive particles, processes like Mechano-Fusion and Cyclomixing being clearly preferred. The collisions between the particles in a jet mill are somewhat uncontrolled and those skilled in the art, therefore, considered it unlikely for this technique to be able to provide the desired deposition of a coating of additive material on the surface of the active particles. Moreover, it was believed that, unlike the situation with Mechano-Fusion and Cyclomixing, segregation of the powder constituents occurred in jet mills, such that the finer particles, that were believed to be the most effective, could escape from the process. In contrast, it could be clearly envisaged how techniques such as Mechano-Fusion would result in the desired coating.

It should also be noted that it was also previously believed that the compressive or impact milling processes must be carried out in a closed system, in order to prevent segregation of the different particles. This has also been found to be untrue and the co-jet milling processes according to the present invention do not need to be carried out in a closed system. Even in an open system, the co-jet milling has surprisingly been found not to result in the loss of the small particles, even when using leucine as the additive material.

It has now unexpectedly been discovered that composite particles of active and additive material can be produced by co-jet milling these materials. The resultant particles have excellent characteristics which lead to greatly improved performance when the particles are dispensed from a DPI for administration by inhalation. In particular, co-jet milling active and additive particles can lead to further significant particle size reduction. What is more, the composite active particles exhibit an enhanced FPD and FPF, compared to those disclosed in the prior art.

The effectiveness of the promotion of dispersal of active particles has been found to be enhanced by using the co-jet milling methods according to the present invention in comparison to compositions which are made by simple blending of similarly sized particles of active material with additive material. The phrase “simple blending” means blending or mixing using conventional tumble blenders or high shear mixing and basically the use of traditional mixing apparatus which would be available to the skilled person in a standard laboratory.

In another embodiment, the particles produced using the two-step process discussed above subsequently undergo Mechano-Fusion. This final Mechano-Fusion step is thought to “polish” the composite active particles, further rubbing the additive material into the particles. This allows one to enjoy the beneficial properties afforded to particles by Mechano-Fusion, in combination with the very small particles sizes made possible by the co-jet milling.

The size of the intranasal doses of methotrexate can vary from micrograms to tens of milligrams. In one embodiment of the invention, the composition is intended for once a week administration and the dose of methotrexate is preferably between 5 μg and 3000 μg, or between 25 μg and 500 μg.

In an alternative embodiment, the composition is intended for daily administration and the dose of methotrexate is preferably between 1 μg and 500 μg, or between 5 μg and 100 μg. When administered daily, the dose of methotrexate may be given in a single dose or divided into up to 4 doses.

Methotrexate may be administered intranasally using a range of devices, including multi- and single-dose pumps such as those manufactured by Valois, Kurve Technology, Inc's ViaNase™ device and the OptiNose system.

Whether intended for administration by inhalation or intranasally, the dry powder compositions of the present invention may benefit from including particles of methotrexate (and any other pharmaceutically active material included) which are relatively dense particles. Thus, powders according to some embodiments of the present invention may preferably have a tapped density of more than 0.1 g/cc, more than 0.2 g/cc, more than 0.3 g/cc, more than 0.4 g/cc, or more than 0.5 g/cc. The inclusion of such relatively dense particles of active material in dry powder compositions unexpectedly leads to good FPFs and FPDs and these dense particles may help reduce the volume of powder that must be administered to the lung or nasal mucosa. Especially in the case of intranasal administration, keeping the volume of powder to a minimum is beneficial, as it can help to reduce any discomfort felt by the patient.

Embodiments of the present invention are further explained by the following examples.

### PASSIVE DPIS

### Example 1

### Mechanofused Methotrexate with Magnesium Stearate

This example studies magnesium stearate processed with micronised methotrexate powder. The blends are prepared by Mechanofusion using the Hosokawa AMS-MINI, with blending being carried out for 60 minutes at approximately 4000 rpm. The magnesium stearate used is a standard pharmaceutical vegetable grade.

Blends of methotrexate and magnesium stearate are prepared at different weight percentages of magnesium stearate. Blends of 5% w/w and 10% w/w, are prepared and then loaded into gelatine capsules and fired from the Miat Monohaler inhaler.

### Example 2

### Mechanofused Methotrexate with Fine Lactose and Magnesium Stearate

A further study is conducted to look at the Mechanofusion of a drug with both a force control agent and fine lactose particles. The additive or force control agent used is magnesium stearate (Peter Greven) and the fine lactose is Sorbolac 400 (Meggle). The drug used is micronised methotrexate.

The blends are prepared by Mechanofusion of all three components together using the Hosokawa AMS-MINI, blending is carried out for 60 minutes at approximately 4000 rpm.

Formulations are prepared using the following concentrations of methotrexate, magnesium stearate and Sorbolac 400:
5% w/w methotrexate, 6% w/w magnesium stearate, 89% w/w Sorbolac 400;
20% w/w methotrexate, 5% w/w magnesium stearate, 75% w/w Sorbolac 400;
20% w/w methotrexate, 2% w/w magnesium stearate, 78% w/w Sorbolac 400.

Blends are then loaded into HPMC capsules and fired from the Miat Monohaler inhaler.

As an extension to this work, different blending methods of methotrexate, magnesium stearate and Sorbolac 400 are investigated further. Two formulations are prepared in the Glen Creston Grindomix. This mixer is a conventional food-processor style bladed mixer, with 2 parallel blades.

The first of these formulations is a 5% w/w methotrexate, 6% w/w magnesium stearate, 89% w/w Sorbolac 400 blend prepared by mixing all components together at 2000 rpm for 20 minutes. The second formulation is a blend of 90% w/w of mechanofused magnesium stearate: Sorbolac 400 (5:95) pre-blend and 10% w/w methotrexate blended in the Grindomix for 20 minutes. It is also observed that this formulation has notably good flow properties for a material comprising such fine particles. This is believed to be associated with the Mechanofusion process.

In a further study, these blends of drug and FCA or drug, fine lactose and FCA are further added to a large lactose carrier to improve the powder flow still further. The large lactose carrier could be the Crystalac or Prismalac grade, for example.

### Example 3

### Preparation of Mechanofused Formulation for Use in a Passive Device

20 g of a mix comprising 20% micronised methotrexate, 78% Sorbolac 400 (fine lactose) and 2% magnesium stearate are weighed into the Hosokawa AMS-MINI Mechanofusion system via a funnel attached to the largest port in the lid with the equipment running at 3.5%. The port is sealed and the cooling water switched on. The equipment is run at 20% for 5 minutes followed by 80% for 10 minutes. The equipment is switched off, dismantled and the resulting formulation recovered mechanically.

20 mg of the collected powder formulation is filled into a blister strip and fired from a Gyrohaler.

### Example 4

### Mechanofused Methotrexate and Mechanofused Fine Lactose

Firstly, 20 g of a mix comprising 95% micronised methotrexate and 5% magnesium stearate are weighed into the Hosokawa AMS-MINI Mechanofusion system via a funnel attached to the largest port in the lid with the equipment running at 3.5%. The port is sealed and the cooling water switched on. The equipment is run at 20% for 5 minutes followed by 80% for 10 minutes. The equipment is then switched off, dismantled and the resulting formulation recovered mechanically.

Next, 20 g of a mix comprising 99% Sorbolac 400 lactose and 1% magnesium stearate are weighed into the Hosokawa AMS-MINI Mechanofusion system via a funnel attached to the largest port in the lid with the equipment running at 3.5%. The port is sealed and the cooling water switched on. The equipment is run at 20% for 5 minutes followed by 80% for 10 minutes. The equipment is switched off, dismantled and the resulting formulation recovered mechanically.

4 g of the methotrexate-based material and 16 g of the Sorbolac-based material are combined in a high shear mixer for 10 minutes, to form the final formulation. 20 mg of the powder formulation are filled into size 3 capsules and fired from a Miat Monohaler into an NGI.

### Example 5

### Jet Milled Methotrexate and Mechanofused Fine Lactose

20 g of a mix comprising 95% micronised methotrexate and 5% magnesium stearate are co-jet milled in a Hosokawa AS50 jet mill.

20 g of a mix comprising 99% Sorbolac 400 (fine lactose) and 1% magnesium stearate are weighed into the Hosokawa AMS-MINI Mechanofusion system via a funnel attached to the largest port in the lid with the equipment running at 3.5%. The port is sealed and the cooling water switched on. The equipment is run at 20% for 5 minutes followed by 80% for 10 minutes. The equipment is switched off, dismantled and the resulting formulation recovered mechanically. 4 g of the methotrexate-based material and 16 g of the Sorbolac-based material are combined in a high shear mixer for 10 minutes, to form the final formulation. 20 mg of the powder formulation are filled into size 3 capsules and fired from a Miat Monohaler into an NGI.

The results of these experiments are expected to show that the powder formulations prepared using the method according to the present invention exhibit further improved properties such as FPD, FPF, as well as good flow and reduced device retention and throat deposition.

### Example 6

### Active DPI Examples

10.0 g of Sorbolac 400 lactose, 10.0 g of methotrexate and 1.0 g of micronised L-leucine were combined in the MechanoFusion system. The material is processed at a setting of 20% power for 5 minutes, followed by a setting of 80% power for 10 minutes. This material is recovered and recorded as “A”.

2.1 g methotrexate plus 0.4 g micronised leucine and 2.5 g micronised lactose are blended. This mixture is then processed in the AS50 Spiral jet mill using an inlet pressure of 7 bar and a grinding pressure of 5 bar, feed rate 5 ml/min. This powder is gently pushed through a 300 μm metal sieve with a spatula. This material is recorded as “B”.

9 g micronised methotrexate plus 1 g micronised leucine are processed in the AS50 Spiral jet mill using an inlet pressure of 7 bar and a grinding pressure of 5 bar, feed rate 5 ml/min. This material is recorded as “C”.

In examples A to C, the process conditions may be varied, and the leucine replaced with other FCAs such as magnesium stearate or lecithin.

A number of foil blisters are filled with approximately 2 mg of the formulations A to C. These are then fired from an Aspirair device into an NGI at a flow rate of 60 l/m.

The % w/w of additive material will typically vary. Firstly, when the additive material is added to the drug, the amount used is preferably in the range of 0.1% to 50%, more preferably 10% to 20%, more preferably 2% to 10%, and most preferably 3 to 8%. Secondly, when the additive material is added to the carrier particles, the amount used is preferably in the range of 0.01% to 30%, more preferably of 0.1% to 10%, preferably 0.2% to 5%, and most preferably 0.5% to 2%. The amount of additive material preferably used in connection with the carrier particles will be heavily dependant upon the size and hence surface area of these particles.

### Example 7

### Methotrexate Mechanofused pMDI Suspension

### Powder Preparation:

12.0 g micronised methotrexate and 4.0 g lecithin S PC-3 (Lipoid) are weighed into a beaker. The powder is transferred to the Hosokawa AMS-MINI via a funnel attached to the largest port in the lid with the equipment running at 3.5%. The port is sealed and the cooling water switched on. The equipment is run at 20% for 5 minutes followed by 50% for 10 minutes. The equipment is switched off, dismantled and the resulting formulation recovered mechanically.

### Preparation of Cans:

0.05 g of powder are weighed into a canister, a 50 μl Bespak valve is crimped to the can and 12.2 g HFA 134a are injected under pressure. The canister is placed in an ultrasonic bath and sonicated for 10 minutes.

Alternatively, other known solution-based or suspension based methods could be used to prepare alternative pMDI-based methotrexate inhalers.

1. A composition comprising methotrexate, wherein the composition is for pulmonary or intranasal administration to provide a therapeutic effect.

2. A composition as claimed in claim 1, for use in treating inflammation.

3. A composition as claimed in claim 1, wherein the composition further comprises an anti-inflammatory agent.

4. A composition as claimed in claim 3, wherein the anti-inflammatory agent has a complementary mechanism of action.

5. A composition as claimed in claim 2, wherein the inflammation is of the upper and/or lower airways.

6. A composition as claimed in claim 5, wherein the inflammation is associated with a chronic respiratory disease such as sarcoidosis, chronic obstructive pulmonary disease (COPD), cystic fibrosis (CF) or asthma.

7. A composition as claimed in claim 6, wherein the chronic respiratory disease is associated with evidence of systemic inflammation.

8. A composition as claimed in claim 7, wherein the evidence of systemic inflammation is an elevated concentration of C-reactive protein (CRP) in the plasma, or the expression of inflammation-related genes such as IL1β, in peripheral blood neutrophils ex vivo.

9. A composition as claimed in claim 1, wherein the composition further comprises a bronchodilator.

10. A composition as claimed in claim 1, wherein the composition is suitable for administration by inhalation.

11. A composition as claimed in claim 1, wherein the inflammation is associated with sleep apnoea.

12. A composition as claimed in claim 11, wherein the composition is suitable for intranasal administration.

13. A composition as claimed in claim 1, wherein the composition is a dry powder.

14. A composition as claimed in claim 13, wherein the powder comprises an additive material which is a force control agent.

15. A composition as claimed in claim 13, wherein the powder further comprises carrier particles.

16. A composition as claimed in claim 13, wherein the powder has a fine particle fraction (<5 μm) of at least 50.

17. A composition as claimed in claim 1, wherein composition is a solution.

18. A composition as claimed in claim 16, wherein composition is formulated for delivery using a pressurised metered dose inhaler.

19. A composition as claimed in claim 17, wherein the composition comprises a propellant.

20. A composition as claimed in claim 17, wherein composition is formulated for delivery using a nebuliser or soft mist inhaler.

21. A composition as claimed in claim 1, further comprising a solvent and/or water.

22. A composition as claimed in claim 1, wherein the composition is intended for once a week administration and the dose of methotrexate is between 5 μg and 3000 μg.

23. A composition as claimed in claim 1, wherein the composition is intended for daily administration and the dose of methotrexate is between 1 μg and 500 μg.

24. A composition as claimed in claim 23, wherein the daily dose of methotrexate is given in a single dose or is divided into up to 4 doses.

25. A composition as claimed in claim 1, wherein the composition is for administration with oral folic acid rescue therapy.

26. The composition of claim 4, wherein the anti-inflammatory is a corticosteroid or PDEIV inhibitor.

27. The composition of claim 9, wherein the bronchodilator is a β₂ agonist or an antimuscarinic agent.

28. The composition of claim 16, wherein the powder has a fine particle fraction (<5 μm) of at least 60%.

29. The composition of claim 16, wherein the powder has a fine particle fraction (<5 μm) of at least 70%.

30. The composition of claim 16, wherein the powder has a fine particle fraction (<5 μm) of at least 80%.

31. The composition of claim 22, wherein the dose of methotrexate is between 25 μg and 500 μg.

32. The composition of claim 23, wherein the dose of methotrexate is between 5 μg and 100 μg.
